(19) United States
(12) Patent Application Publication
(10) Pub. No.: US 2008/0086010 A1
(43) Pub. Date: Apr. 10, 2008

(54) Processes for the preparation of 4-chloro-3-methyl-5-(2-(2-(6-methylbenzo[d][1,3]dioxol-5-yl)acetyl)-3-thienylsulfonamido)isoxazole

(76) Inventor: John F. Reichwein, Houston, TX (US)

Correspondence Address: JONES DAY, 222 EAST 41ST ST, NEW YORK, NY, 10017, US

(21) Appl. No.: 11/890,200

(22) Filed: Aug. 3, 2007

Related U.S. Application Data
(60) Provisional application No. 60/835,635, filed on Aug. 4, 2006.

(51) Int. Cl. C07D 407/02; C07D 513/04

(52) U.S. Cl. 548/210; 548/246

(57) ABSTRACT

Provided are processes for the preparation of 4-chloro-3-methyl-5-(2-(2-(6-methylbenzo[d][1,3]dioxol-5-yl)acetyl)-3-thienylsulfonamido)isoxazole, a compound useful for the treatment of endothelin-mediated disorders.

### RELATED APPLICATION

Priority is claimed herein under 35 U.S.C. §119(e) to U.S. Provisional Patent Application Ser. No. 60/835,635, filed Aug. 4, 2006, entitled “PROCESSES FOR THE PREPARATION OF 4-CHLORO-3-METHYL-5-(2-(2-(6-METHYLBENZO[D][1,3]-DIOXOL-5-YL)ACETYL)-3-THIENYLSULFONAMIDO)ISOXAZOLE.” The disclosure of the above-referenced application is incorporated by reference herein in its entirety.

### FIELD

Provided herein are processes for the preparation of 4-chloro-3-methyl-5-(2-(2-(6-methylbenzo[d][1,3]dioxol-5-yl)acetyl)-3-thienylsulfonamido)isoxazole, a compound useful for the treatment of endothelin-mediated disorders.

### BACKGROUND

4-chloro-3-methyl-5-(2-(2-(6-methylbenzo[d][1,3]dioxol-5-yl)acetyl)-3-thienylsulfonamido)isoxazole is an endothelin antagonist. (See U.S. Pat. Nos. 5,783,705, 5,962,490, 6,248,767) Endothelin antagonists are useful for the treatment of hypertension such as peripheral circulatory failure, heart disease such as angina pectoris, cardiomyopathy, arteriosclerosis, myocardial infarction, pulmonary hypertension, vasospasm, vascular restenosis, Raynaud's disease, cerebral stroke such as cerebral arterial spasm, cerebral ischemia, late phase cerebral spasm after subarachnoid hemorrhage, asthma, bronchoconstriction, renal failure, particularly post-ischemic renal failure, cyclosporine nephrotoxicity such as acute renal failure, colitis, as well as other inflammatory diseases, endotoxic shock caused by or associated with endothelin, and other diseases in which endothelin has been implicated. Provided herein are processes for the preparation of 4-chloro-3-methyl-5-(2-(2-(6-methylbenzo[d][1,3]dioxol-5-yl)acetyl)-3-thienylsulfonamido)isoxazole.

### SUMMARY

Provided herein are processes for the preparation of 4-chloro-3-methyl-5-(2-(2-(6-methylbenzo[d][1,3]dioxol-5-yl)acetyl)-3-thienylsulfonamido)isoxazole: 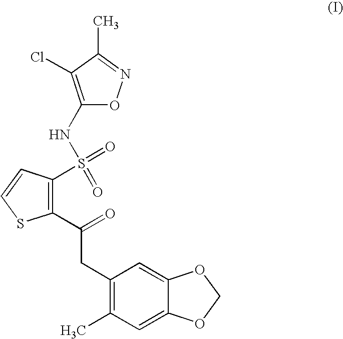
or a pharmaceutically acceptable derivative thereof.

In one aspect, provided is a process for preparing the compound of Formula (I), or a pharmaceutically acceptable derivative thereof, wherein the process involves a step of reacting a compound of Formula (II): 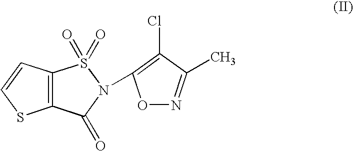
with a compound of Formula (III): 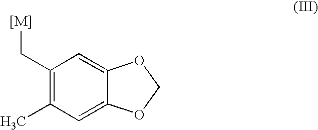
wherein [M] is a metal, a metal halide, or a metal complex.

### DETAILED DESCRIPTION

### Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein, pharmaceutically acceptable derivatives of a compound include salts, esters, enol ethers, enol esters, acetals, ketals, orthoesters, hemiacetals, hemiketals, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, alk(en)(yn)yl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, alk(en)(yn)yl, aryl, aralkyl, or cycloalkyl. Pharmaceutically acceptable enol esters include, but are not limited to, derivatives of formula C═C(OC(O)R) where R is hydrogen, alkyl, alkenyl, alkynyl, aryl, aralkyl, or cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, alkyl refers to an unbranched or branched hydrocarbon chain. An alkyl group may be unsubstituted or substituted with one or more heteroatoms.

As used herein, alkenyl refers to an unbranched or branched hydrocarbon chain comprising one or more double bonds. The double bond of an alkenyl group may be unconjugated or conjugated to another unsaturated group. An alkenyl group may be unsubstituted or substituted with one or more heteroatoms.

As used herein, alkynyl refers to an unbranched or branched hydrocarbon chain comprising one of more triple bonds therein. The triple bond of an alkynyl group may be unconjugated or conjugated to another unsaturated group. An alkynyl group may be unsubstituted or substituted with one or more heteroatoms.

As used herein, alk(en)(yn)yl refers to an unbranched or branched hydrocarbon group comprising at least one double bond and at least one triple bond. The double bond or triple bond of an alk(en)(yn)yl group may be unconjugated or conjugated to another unsaturated group. An alk(en)(yn)yl group may be unsubstituted or substituted with one or more heteroatoms.

Exemplary alkyl, alkenyl, alkynyl, and alk(en)(yn)yl groups herein include, but are not limited to, methyl, ethyl, propyl, isopropyl, isobutyl, n-butyl, sec-butyl, tert-butyl, isopentyl, neopentyl, tert-pentyl, isohexyl, allyl(propenyl) and propargyl(propynyl).

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl or isoquinolinyl.

As used herein, “halo,” “halogen,” or “halide” refers to F, Cl, Br or I.

As used herein, solvent refers to any liquid that completely or partially dissolves a solid, liquid, or gaseous solute, resulting in a solution such as but not limited to hexane, benzene, toluene, diethyl ether, chloroform, ethyl acetate, dichloromethane, carbon tetrachloride, 1,4-dioxane, tetrahydrofuran, glyme, diglyme, acetone, acetonitrile, dimethylformamide, dimethyl sulfoxide, dimethylacetamide, or N-methyl-2-pyrrolidone.

As used herein, dehydrating agent refers to any compound that promotes the formation of carboxamides from carboxylic acids, such as but not limited to thionyl chloride, sulfuryl chloride, a carbodiimide, an anhydride or a mixed anhydride, a phenol (such as but not limited to nitrophenol, pentafluorophenol, or phenol), or a compound of Formula (A): 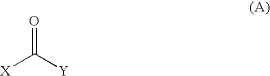
wherein each of X and Y is independently an unsubstituted or substituted heteroaryl group (such as but not limited to imidazolyl, benzimidazolyl, or benzotriazolyl). Examples of dehydrating agents further include, but are not limited to, benzotriazole-1-yl-oxy-tris-(dimethylamino)-phosphonium hexafluorophosphate (BOP), N,N′-carbonyldiimidazole (CDI), 3-(diethoxyphosphoryloxy)-1,2,3-benzotriazin-4(3H)-one (DEPBT), 1-ethyl-3-(3-dimethyllaminopropyl)carbodiimide (EDC), 2-(7-aza-1H-benzotriazole-1-yl)-1,1,3,3-tetramethyluronium hexafluorophosphate (HATU), 2-(1H-benzotriazole-1-yl)-1,1,3,3-tetramethyluronium hexafluorophosphate (HBTU), 1-hydroxybenzotriazole (HOBt), benzotriazole-1-yl-oxy-tris-pyrrolidino-phosphonium hexafluorophosphate (PyBOP), 2-(1H-benzotriazole-1-yl)-1,1,3,3-tetramethyluronium tetrafluoroborate (TBTU), O-(3,4-dihydro-4-oxo-1,2,3-benzotriazine-3-yl)-N,N,N,N-tetramethyluronium tetrafluoroborate (TDBTU), 3-(diethyloxyphosphoryloxy)-1,2,3-benzotriazin-4(3H)-one (DEPBT), dicyclohexylcarbodiimide (DCC), N,N′-diisopropylcarbodiimide (DIC), or 1-hydroxy-7-azabenzotriazole (HOAt).

As used herein, metal complex refers to any metal that contains one or more ligand(s). It is to be understood that metal complexes include but are not limited to coordination complexes (including but not limited to organometallic complexes, Werner complexes, clusters, bioinorganic complexes, and combinations thereof).

It is to be understood that reactants, compounds, solvents, acids, bases, catalysts, agents, reactive groups, or the like may be added individually, simultaneously, separately, and in any order. Furthermore, it is to be understood that reactants, compounds, acids, bases, catalysts, agents, reactive groups, or the like may be pre-dissolved in solution and added as a solution (including, but not limited to, aqueous solutions). In addition, it is to be understood that reactants, compounds, solvents, acids, bases, catalysts, agents, reactive groups, or the like may be in any molar ratio.

It is to be understood that reactants, compounds, solvents, acids, bases, catalysts, agents, reactive groups, or the like may be formed in situ.

Processes

Provided herein is a process for preparing a compound of Formula (I) or a pharmaceutically acceptable derivative thereof involving the step of reacting a compound of Formula (II) with a compound of Formula (III) as depicted in Scheme A below, wherein [M] is a metal, metal halide, or metal complex.

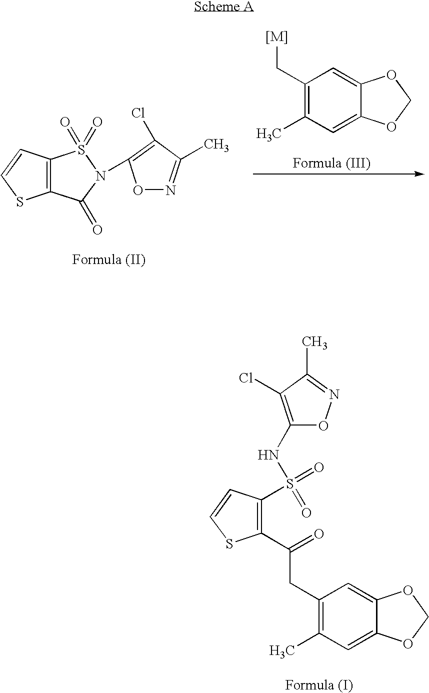

In some embodiments, [M] contains Mg, Li, Zn, Sm, In, Sn, Ca, or Mn. In some embodiments, [M] is MgBr, MgCl, MgI, Li, ZnBr, ZnCl, or ZnI.

The compound of Formula (III), wherein [M] is a metal, a metal halide, or a metal complex may be prepared using methods known to those of ordinary skill in the art. For example, the preparation of the Grignard reagent of the compound of Formula (III), wherein [M] is MgCl, has been reported by Wu et al. (U.S. Pat. No. 6,683,103 B2).

The preparation of the compound of Formula (I) or a pharmaceutically acceptable derivative thereof as depicted in Scheme A may occur in any solvent or any combination of solvents. In some embodiments, the solvent is, or the combination of solvents contains, diethyl ether, 1,4-dioxane, tetrahydrofuran, dimethylformamide, dimethyl sulfoxide, glyme, diglyme, dimethylacetamide, or N-methyl-2-pyrrolidone. In some embodiments, the solvent is tetrahydrofuran.

In some embodiments of the preparation of the compound of Formula (I) or a pharmaceutically acceptable derivative thereof as depicted in Scheme A, [M] is MgCl and the solvent is tetrahydrofuran.

The preparation of the compound of Formula (I) or a pharmaceutically acceptable derivative thereof as depicted in Scheme A may occur at any reaction temperature. In some embodiments, the reaction temperature may vary from about −100° C. to about 100° C. In some embodiments, the reaction temperature may vary from about −50° C. to about 50° C. In some embodiments, the reaction temperature may vary from about −10° C. to about 10° C. In some embodiments, the reaction temperature may vary from about −85° C. to about −75° C. In some embodiments, the reaction temperature is about −78° C.

In some embodiments of the preparation of the compound of Formula (I) or a pharmaceutically acceptable derivative thereof as depicted in Scheme A, [M] is MgCl, the solvent is tetrahydrofuran, and the reaction temperature is about −78° C.

The preparation of the compound of Formula (I) or a pharmaceutically acceptable derivative thereof as depicted in Scheme A may occur at any reaction time. In some embodiments, the reaction time is from about 1 minute to about 24 hours. In some embodiments, the reaction time is from about 1 minute to about 8 hours. In some embodiments, the reaction time is from about 1 minute to about 3 hours. In some embodiments, the reaction time is from about 1 minute to about 1 hour. In some embodiments, the reaction time is about 5 minutes.

In some embodiments of the preparation of the compound of Formula (I) or a pharmaceutically acceptable derivative thereof as depicted in Scheme A, [M] is MgCl, the solvent is tetrahydrofuran, the reaction temperature is about −78° C., and the reaction time is about 5 minutes.

The preparation of the compound of Formula (I) as depicted in Scheme A may occur at any molar ratio according to a person of ordinary skill in the art. In some embodiments, the molar ratio of the compound of Formula (II) to the compound of Formula (III) is from about 10:1 to about 1:1. In some embodiments, the molar ratio of the compound of Formula (II) to the compound of Formula (III) is from about 5:1 to about 1:1. In some embodiments, the molar ratio of the compound of Formula (II) to the compound of Formula (III) is from about 2:1 to about 1:1. In some embodiments, the molar ratio of the compound of Formula (II) to the compound of Formula (III) is about 1.25:1. In some embodiments, the molar ratio of the compound of Formula (III) to the compound of Formula (II) is from about 10:1 to about 1:1. In some embodiments, the molar ratio of the compound of Formula (III) to the compound of Formula (II) is from about 5:1 to about 1:1. In some embodiments, the molar ratio of the compound of Formula (III) to the compound of Formula (II) is from about 3:1 to about 1:1.

In some embodiments of the preparation of the compound of Formula (I) or a pharmaceutically acceptable derivative thereof as depicted in Scheme A, [M] is MgCl, the solvent is tetrahydrofuran, the reaction temperature is about −78° C., the reaction time is about 5 minutes, and the molar ratio of the compound of Formula (II) to the compound of Formula (III) is about 1.25:1.

In another embodiment, provided is a process for preparing a compound of Formula (II) or a pharmaceutically acceptable derivative thereof by reacting a compound of Formula (IV) with a dehydrating agent or a combination of dehydrating agents as depicted in Scheme B.

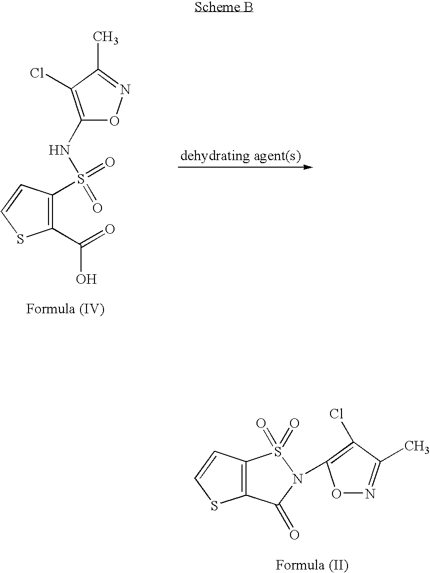

In another embodiment, provided is a process for preparing a compound of Formula (II) or a pharmaceutically acceptable derivative thereof by reacting a compound of Formula (V) with a dehydrating agent or a combination of dehydrating agents as depicted in Scheme C.

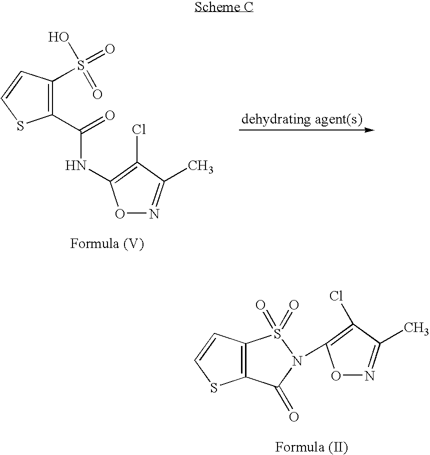

The compound of Formula (IV) and the compound of Formula (V) may be prepared using methods known to those of ordinary skill in the art. For example, the preparation of the compound of Formula (IV) has been reported in Wu et al, J. Med. Chem. 1999, 42, 4485-4499. The preparation of the compound of Formula (V) may be prepared using similar methods.

The preparation of the compound of Formula (II) or a pharmaceutically acceptable derivative thereof as depicted in Scheme B or Scheme C may occur with any dehydrating agent or any combination of dehydrating agents according to a person of ordinary skill in the art. In some embodiments, the dehydrating agent is (or the combination of dehydrating agents are) generated in situ. In some embodiments, the dehydrating agent is (or the combination of dehydrating agents contains) thionyl chloride, sulfuryl chloride, 4-dimethylaminopyridine, a carbodiimide, an anhydride or a mixed anhydride, a phenol, or a compound of Formula (A): 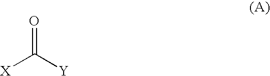
wherein each of X and Y is independently an unsubstituted or substituted heteroaryl group. In some embodiments, the dehydrating agent is (or combination of dehydrating agents contains) benzotriazole-1-yl-oxy-tris-(dimethylamino)-phosphonium hexafluorophosphate (BOP), N,N′-carbonyldiimidazole (CDI), 3-(diethoxyphosphoryloxy)-1,2,3-benzotriazin-4(3H)-one (DEPBT), 1-ethyl-3-(3-dimethyllaminopropyl)carbodiimide (EDC), 2-(7-aza-1H-benzotriazole-1-yl)-1,1,3,3-tetramethyluronium hexafluorophosphate (HATU), 2-(1H-benzotriazole-1-yl)-1,1,3,3-tetramethyluronium hexafluorophosphate (HBTU), 1-hydroxybenzotriazole (HOBt), benzotriazole-1-yl-oxy-tris-pyrrolidino-phosphonium hexafluorophosphate (PyBOP), 2-(1H-benzotriazole-1-yl)-1,1,3,3-tetramethyluronium tetrafluoroborate (TBTU), O-(3,4-dihydro-4-oxo-1,2,3-benzotriazine-3-yl)-N,N,N,N-tetramethyluronium tetrafluoroborate (TDBTU), 3-(diethyloxyphosphoryloxy)-1,2,3-benzotriazin-4(3H)-one (DEPBT), dicyclohexylcarbodiimide (DCC), N,N′-diisopropylcarbodiimide (DIC), or 1-hydroxy-7-azabenzotriazole (HOAt). In some embodiments, the dehydrating agent is dicyclohexylcarbodiimide.

The preparation of the compound of Formula (II) or a pharmaceutically acceptable derivative thereof as depicted in Scheme B or Scheme C may occur in any solvent or any combination of solvents. In some embodiments, the solvent is, or the combination of solvents contains, hexane, benzene, toluene, diethyl ether, chloroform, ethyl acetate, dichloromethane, carbon tetrachloride, 1,4-dioxane, tetrahydrofuran, glyme, diglyme, acetone, acetonitrile, dimethylformamide, dimethyl sulfoxide, dimethylacetamide, or N-methyl-2-pyrrolidone. In some embodiments, the combination of solvents contains ethyl acetate and dichloromethane.

In some embodiments of the preparation of the compound of Formula (II) or a pharmaceutically acceptable derivative thereof as depicted in Scheme B or Scheme C, the dehydrating agent is dicyclohexylcarbodiimide and the combination of solvents contains ethyl acetate and dichloromethane.

The preparation of the compound of Formula (II) or a pharmaceutically acceptable derivative thereof as depicted in Scheme B or Scheme C may occur at any reaction temperature according to a person of ordinary skill in the art. In some embodiments, the reaction temperature is from about 0° C. to about 100° C. In some embodiments, the reaction temperature is from about 20° C. to about 80° C. In some embodiments, the reaction temperature is from about 40° to about 60° C. In some embodiments, the reaction temperature is from about 20° C. to about 30° C.

In some embodiments of the preparation of the compound of Formula (II) or a pharmaceutically acceptable derivative thereof as depicted in Scheme B or Scheme C, the dehydrating agent is dicyclohexylcarbodiimide, the combination of solvents contains ethyl acetate and dichloromethane, and the reaction temperature is from about 20° C. to about 30° C.

The preparation of the compound of Formula (II) or a pharmaceutically acceptable derivative thereof as depicted in Scheme B or Scheme C may occur at any reaction time. In some embodiments, the reaction time is from about 30 minutes to about 24 hours. In some embodiments, the reaction time is from about 6 hours to about 18 hours. In some embodiments, the reaction time is about 16 hours.

In some embodiments of the preparation of the compound of Formula (II) or a pharmaceutically acceptable derivative thereof as depicted in Scheme B or Scheme C, the dehydrating agent is dicyclohexylcarbodiimide, the combination of solvents contains ethyl acetate and dichloromethane, the reaction temperature is from about 20° C. to about 30° C., and the reaction time is about 16 hours.

The preparation of the compound of Formula (II) or a pharmaceutically acceptable derivative thereof as depicted in Scheme B or Scheme C may occur at any molar ratio. In some embodiments, the molar ratio of dicyclohexylcarbodiimide to the compound of Formula (IV) is from about 10:1 to about 1:1. In some embodiments, the molar ratio of dicyclohexylcarbodiimide to the compound of Formula (IV) is from about 5:1 to about 1:1. In some embodiments, the molar ratio of dicyclohexylcarbodiimide to the compound of Formula (IV) is from about 3:1 to about 1:1. In some embodiments, the molar ratio of dicyclohexylcarbodiimide to the compound of Formula (IV) is about 1:1. In some embodiments, the molar ratio of the compound of Formula (IV) to dicyclohexylcarbodiimide is from about 5:1 to about 1:1.

In some embodiments of the preparation of the compound of Formula (II) or a pharmaceutically acceptable derivative thereof as depicted in Scheme B or Scheme C, the dehydrating agent is dicyclohexylcarbodiimide, the combination of solvents contains ethyl acetate and dichloromethane, the reaction temperature is from about 20° C. to about 30° C., the reaction time is about 16 hours, and the molar ratio of dicyclohexylcarbodiimide to the compound of Formula (IV) is about 1:1.

The following examples are provided for illustration only and are not intended to limit the scope of this disclosure.

### EXAMPLE 1

### Preparation of the Compound of Formula (II)

To a solution of 20.0 g of the compound of Formula (IV) in 200 ml EtOAc and 100 ml DCM was added 13.0 g DCC. After stirring for 16 hours the reaction mixture was filtered through a silica gel plug and the silica gel plug was washed with EtOAc, followed by concentration in vacuo. The crude was recrystallized form 400 ml hot EtOAC:Hexanes (1:1) go give 13.8 g of the title compound has on off white crystalline material. ¹H NMR (400 MHz, CDCl₃): δ 2.24 (s, 3H), 7.32 (d, J=5.1 Hz, 1H), 7.83 (d, J=5.1 Hz, 1H) ppm. MS (ESI) m/z: 304.96 [M+H]⁺.

### EXAMPLE 2

### Preparation of the Compound of Formula (I)

To a −78° C. solution of 700 mg of the compound of Formula (II) in 10 ml THF under a nitrogen atmosphere was slowly added 3.5 ml 0.53 M solution of the compound of Formula (III) ([M]=MgCl) in THF. The cooling bath was removed. After stirring for 5 minutes under a nitrogen atmosphere 10 ml 2N HCl was added, followed by extraction with 25 ml toluene. The organic layer was washed with 25 ml 2N HCl, followed by extraction with 2×25 ml sat. NaHCO₃. The bicarbonate layers were combined and extracted with 2×25 ml EtOAc. The organic layers were combined and washed with 2N HCl/brine solution, dried over MgSO₄ and concentrated in vacuo. The crude was reconstituted in 50% water-MeCN and purified by preparative HPLC on a 30×250 mm Waters Symmetry Shield 7 μm RP 18 column with 3 injections using 35% to 75% B as gradient (A: 0.1% TFA in water; B: MeCN; diluent: 50% water in MeCN). The product containing fractions were combined and lyophilized to yield 12 mg of the title compound as a pale yellow solid in >99% purity.

Since modifications will be apparent to those of skill in the art, this disclosure is intended to be limited only by the scope of the appended claims.

What is claimed:

1. A process for preparing a compound of Formula (I): 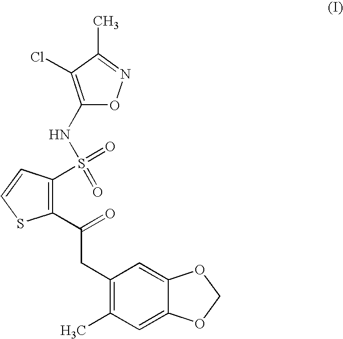
or a pharmaceutically acceptable derivative thereof, comprising a step of reacting a compound of Formula (II): 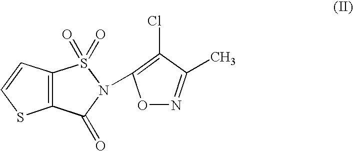
with a compound of Formula (III): 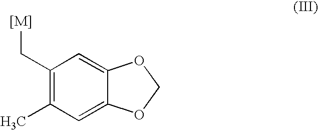
wherein [M] is a metal, a metal halide, or a metal complex.

2. The process of claim 1, wherein [M] comprises Mg, Li, Zn, Sm, In, Sn, Ca, or Mn.

3. The process of claim 1, wherein [M] is MgBr, MgCl, MgI, Li, ZnBr, ZnCl, ZnI.

4. The process of claim 1, wherein [M] is MgCl.

5. The process of claim 1, wherein the reaction occurs in a solvent or a combination of solvents.

6. The process of claim 1, wherein the solvent is, or the combination of solvents comprises, diethyl ether, 1,4-dioxane, tetrahydrofuran, dimethylformamide, dimethyl sulfoxide, glyme, diglyme, dimethylacetamide, or N-methyl-2-pyrrolidone.

7. The process of claim 1, wherein the solvent is tetrahydrofuran.

8. The process of claim 1, wherein the reaction temperature is from about −100° C. to about 30° C.

9. The process of claim 1, wherein the reaction temperature is about −78° C.

10. The process of claim 1, wherein the reaction time is from about 1 minute to about 24 hours.

11. The process of claim 1, wherein the reaction time is about 5 minutes.

12. The process of claim 1, wherein the molar ratio of the compound of Formula (II) to the compound of Formula (III) is from about 3:1 to about 1:1.

13. The process of claim 1, wherein the molar ratio of the compound of Formula (III) to the compound of Formula (II) is from about 3:1 to about 1:1.

14. The process of claim 1 wherein the molar ratio of the compound of Formula (II) to the compound of Formula (III) is about 1.25:1.

15. The process of claim 1, further comprising preparing the compound of Formula (II) by reacting a compound of Formula (IV): 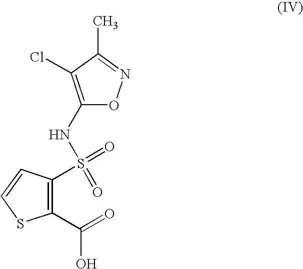
with a dehydrating agent or a combination of dehydrating agents.

16. A process of preparing a compound of Formula (II): 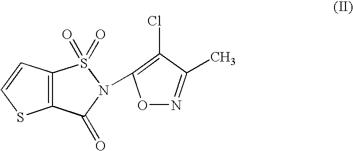
or a pharmaceutically acceptable derivative thereof, comprising the step of reacting a compound of Formula (IV): 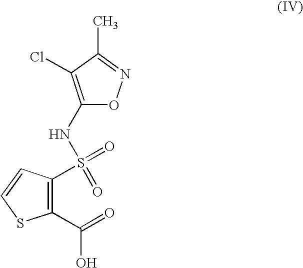
with a dehydrating agent of a combination of dehydrating agents.

17. The process of claim 15, wherein the dehydrating agent is, or the combination of dehydrating agents comprises, thionyl chloride, sulfuryl chloride, a carbodiimide, an anhydride or a mixed anhydride, a phenol, or a compound of Formula (A): 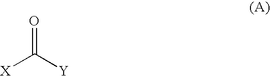
wherein each of X and Y is independently an unsubstituted or substituted heteroaryl group.

18. The process of claim 15, wherein the dehydrating agent is, or the combination of dehydrating agents comprises, benzotriazole-1-yl-oxy-tris-(dimethylamino)-phosphonium hexafluorophosphate, N,N′-carbonyldiimidazole, 3-(diethoxyphosphoryloxy)-1,2,3-benzotriazin-4(3H)-one, 1-ethyl-3-(3-dimethyllaminopropyl)carbodiimide, 2-(7-aza-1H-benzotriazole-1-yl)-1,1,3,3-tetramethyluronium hexafluorophosphate, 2-(1H-benzotriazole-1-yl)-1,1,3,3-tetramethyluronium hexafluorophosphate, 1-hydroxybenzotriazole, benzotriazole-1-yl-oxy-tris-pyrrolidino-phosphonium hexafluorophosphate, 2-(1H-benzotriazole-1-yl)-1,1,3,3-tetramethyluronium tetrafluoroborate, O-(3,4-dihydro-4-oxo-1,2,3-benzotriazine-3-yl)-N,N,N,N-tetramethyluronium tetrafluoroborate, 3-(diethyloxyphosphoryloxy)-1,2,3-benzotriazin-4(3H)-one, dicyclohexylcarbodiimide, N,N′-diisopropylcarbodiimide, or 1-hydroxy-7-azabenzotriazole, nitrophenol, pentafluorophenol, or phenol.

19. The process of claim 15, wherein the dehydrating agent is dicyclohexylcarbodiimide.

20. The process of claim 15, wherein the reaction occurs in a solvent or a combination of solvents.

21. The process of claim 20, wherein the solvent is, or the combination of solvents comprises, hexane, benzene, toluene, diethyl ether, chloroform, ethyl acetate, dichloromethane, carbon tetrachloride, 1,4-dioxane, tetrahydrofuran, glyme, diglyme, acetone, acetonitrile, dimethylformamide, dimethyl sulfoxide, dimethylacetamide, or N-methyl-2-pyrrolidone.

22. The process of claim 20, wherein the combination of solvents comprises ethyl acetate and dichloromethane.

23. The process of claim 15, wherein the reaction temperature is from about 0° C. to about 100° C.

24. The process of claim 15, wherein the reaction temperature is from about 20° C. to about 30° C.

25. The process of claim 15, wherein the reaction time is from about 30 minutes to about 24 hours.

26. The process of claim 15, wherein the reaction time is about 16 hours.

27. The process of claim 15, wherein the molar ratio of dicyclohexylcarbodiimide to the compound of Formula (IV) is from about 5:1 to about 1:1.

28. The process of claim 15, wherein the molar ratio of dicyclohexylcarbodiimide to the compound of Formula (IV) is 1:1.

29. The process of claim 15, wherein the molar ratio of the compound of Formula (IV) to dicyclohexylcarbodiimide is from about 5:1 to about 1:1.

30. A compound of Formula (II): 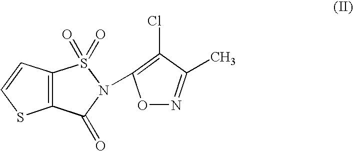
or a pharmaceutically acceptable derivative thereof.
